(19) United States
(12) Patent Application Publication
(10) Pub. No.: US 2009/0238905 A1
(43) Pub. Date: Sep. 24, 2009

(54) INFLAMMATION REDUCING ACTION OF SYNERGISTIC MIXTURES OF BISABOLOL AND GINGER EXTRACTS

(75) Inventors: Martin Gurney, East Hanover, NJ (US); Donna Rosa, Nutley, NJ (US); Deborah Kennison, Wayne, NJ (US); Thomas Mangos, Fort Lee, NJ (US)

Correspondence Address: Gregory A. Nelson;Novak Druce & Quigg LLP, 525 Okeechobee Blvd, Suite 1500, West Palm Beach, FL, 33401, US

(73) Assignee: SYMRISE, Inc., Teterboro, NJ (US)

(21) Appl. No.: 12/407,458

(22) Filed: Mar. 19, 2009

Related U.S. Application Data
(60) Provisional application No. 61/038,248, filed on Mar. 20, 2008.

(51) Int. Cl. A61K 36/906; A61P 17/00; A61P 11/00

(52) U.S. Cl. 424/756

(57) ABSTRACT

A formulation for treating or eliminating irritation and/or inflammation-reducing effect on endodermal tissue of the respiratory tract, the gastrointestinal tract, or both is disclosed. The formulation can include bisabolol or an extract containing bisabolol and actives found in ginger or extracted from ginger. The ratio of bisabolol and actives from ginger is adjusted such that the irritation and inflammation-reducing action of the bisabolol constituents and ginger constituents is increased synergistically.

### CROSS-REFERENCE TO RELATED APPLICATIONS

This application claims priority to U.S. Provisional Patent Application No. 61/038,248 filed Mar. 20, 2008, the entirety of which is hereby incorporated by reference.

### STATEMENT REGARDING FEDERALLY SPONSORED RESEARCH OR DEVELOPMENT

N/A

### FIELD OF THE INVENTION

The invention relates to composition for reducing inflammation and irritation of endodermal tissue, such as that found in the gastrointestinal tract and the respiratory tract.

### BACKGROUND OF THE INVENTION

Portions of the respiratory tract and the gastrointestinal tract in the human body involved in absorption and secretion are exposed to the external environment. They are therefore lined with specialized epithelia, which serve as barriers. These linings, which are of mostly endodermal origin, are contiguous with the skin in several places, e.g., at the nostrils, the lips, the ears, the genital area, and the anus. In most cases these linings produce mucus via glands embedded in the epithelium as a protection mechanism. Body cavities featuring those specialized mucus-producing linings include most of the respiratory system and the gastrointestinal tract (“GI tract”).

The respiratory tract is the part of the anatomy that has to do with the process of respiration. It is divided into 3 segments: the upper respiratory tract, which includes the nose and nasal passages, paranasal sinuses, and throat or pharynx; the respiratory airways, which include the voice box or larynx, trachea, bronchi, and bronchioles; and the lungs, which include the respiratory bronchioles, alveolar ducts, alveolar sacs, and alveoli.

The term upper respiratory infections, commonly referred to as URIs, is used to refer to an acute infection that involves the upper respiratory tract, e.g., the nose, sinuses, pharynx or larynx. In the United States, there are approximately one billion acute upper respiratory illnesses annually.

Acute upper respiratory tract infections include rhinosinusitis (common cold), sinusitis, pharyngitis/tonsillitis, laryngitis and sometimes bronchitis. Symptoms of URIs commonly include congestion, cough, running nose, sore throat, fever, facial pressure and sneezing. Onset of the symptoms usually begins 1-3 days after exposure to a microscopic pathogen, most commonly a virus. The duration of the symptoms is typically 7 to 10 days but may persist longer.

A very common infection is pharyngitis. Pharyngitis is, in most cases, a painful inflammation of the pharynx, and is colloquially referred to as a sore throat. Infection of the tonsils, i.e., tonsillitis, may occur simultaneously. About 90% of cases are caused by viral infection, with the remainder caused by bacterial infection and, in rare cases, oral thrush (fungal candidiasis, e.g., in babies). Some cases of pharyngitis are caused by irritation from environmental irritants such as pollutants or chemical substances.

There are three types of treatment for URIs: symptomatic, remedial and preventive. Symptomatic treatments are aimed at reducing pain and symptoms. Remedial treatments attempt to cure pharyngitis by reducing its spread and speeding up the healing process. Preventive treatments attempt to block the start of an infection.

Remedial treatments are mostly effective for bacterial infections such as streptococcal infections. For viral infections, even with treatment, recovery from pharyngitis generally occurs spontaneously within a few days. Hence the most popular method of treatment is symptomatic. Many preventive treatments are also remedial.

Several non-antibiotic treatments for sore throat have been studied in controlled trials. Analgesics are among the most effective treatment, but there are many simple measures that can also be used.

### Symptomatic Treatments

Symptomatic treatments for URIs include:

Analgesics such as NSAIDs can help reduce the pain associated with a sore throat;

Throat lozenges and syrups (cough medicine), films strips and chewing gums are often used for short-term pain relief;

Avoiding foods and liquids highly acidic in nature, as they will provoke temporary periods of intense pain;

Warm tea (true or herbal) or soup can help temporarily alleviate the pain of a sore throat;

Cold beverages, popsicles and ice cubes numb the nerves of the throat somewhat, alleviating the pain of a dry and scratchy throat for a brief time, i.e., non-medicated throat cooler;

Mouthwash (when gargled) reduces the pain but only for a brief time;

Drinking heavy amounts of liquid reduces the pain for a short time;

Peppermint candy or other hard candies, can be used to reduce the pain for a short time, in some cases;

Yogurt and milk have been shown to help alleviate the pain temporarily by coating the affected area;

Gargling with warm saline solution may help reduce mucus, but there is little evidence that it provides any long-term benefit;

Throat sprays for numbing; and

Nasal sprays.

### Remedial and Preventive Treatments

Performing remedial treatments early when the throat begins to feel scratchy may help prevent the infection from spreading to the rest of the throat and back of the mouth, which can result in difficulty swallowing. Treatment should begin the first or second day of the illness, however if a person has a cold or the flu, the infection may still continue to spread to other areas, including the ears through the Eustachian tube (causing an earache) and to the lungs through the trachea (causing a cough). Healthy people who come into frequent contact with individuals with Pharyngitis may also try the measures below as preventive treatments to avoid an infection.

The use of antibiotics is a helpful remedial treatment when a bacterial infection is the cause of the sore throat. For viral sore throats, antibiotics have no effect.

Honey has long been used for treating sore throats due to its antiseptic properties.

Swallowing a couple teaspoons of raw lemon or lime juice several times a day may help destroy microorganisms in bacteria-related throat infections. This remedy should be started during the first or second day of sickness as citric acid can irritate throat tissues after the Pharyngitis becomes widespread. If this is the case, a diluted solution of lemon, honey and tea (or lemon with hot water) may be used.

The gastrointestinal tract (“GI tract”), also called the digestive tract, the alimentary canal, or the entrails, is the system of organs that receives food, digests the food to extract energy and nutrients, and expels the remaining waste. The major functions of the GI tract are ingestion, digestion, absorption, and excretion.

The upper GI tract consists of the mouth, pharynx, esophagus, and stomach. The mouth contains the buccal mucosa, which contains the openings of the salivary glands; the tongue; and the teeth. Behind the mouth lies the pharynx, which leads to a hollow muscular tube, the esophagus, which connects to the stomach. The stomach, in turn, leads to the small intestine. The lower GI tract comprises the intestines and anus. The intestines include the bowel or intestine, the small intestine, which has three parts: duodenum, jejunum, ileum, the large intestine, which has three parts: cecum with the vermiform appendix attached, the colon (ascending colon, transverse colon, descending colon and sigmoid flexure) and the rectum.

Common inflammations of the gastrointestinal tract include gastro-esophageal reflux diseases (GERD), heartburn and peptic ulcers. Physicians first direct treatment to inducing a remission which involves relief of symptoms and healing of the lining of the corresponding membranes and tissues, reducing inflamed tissue and then longer term treatment to maintain the remission.

### Remedial and Preventive Treatments

Chamomile is used medicinally against sore stomach, irritable bowel syndrome, and as a gentle sleep aid. It can be taken as an herbal tea, two teaspoons of dried flower per cup of tea. For a sore stomach, some recommend taking a cup every morning without food for two to three months. The primary active ingredient of the essential oil from German Chamomile is bisabolol.

### SUMMARY OF THE INVENTION

A formulation having an irritation-reducing action, an inflammation reducing action, or both. The formulation includes an ingestible carrier or coating and an active mixture that includes bisabolol or extracts containing bisabolol and a ginger composition. The ginger composition can be selected from: (i) substance mixtures obtainable from an extraction of ginger; (ii) substance mixtures obtainable from a separation of a ginger extract which comprise a compound which is chosen from the group consisting of gingerols, shogaols, gingerdiols, dehydrogingerdiones, paradols and derivatives thereof,; (iii) compounds obtainable from a separation of a ginger extract which are chosen from the group consisting of gingerols, shogaols, gingerdiols, dehydrogingerdiones, paradols and derivatives thereof and mixtures thereof, and (iv) mixtures thereof. The ratio of the bisabolol to the ginger composition in the active mixture is such that an irritation reducing action, an inflammation reducing action, or both, of the bisabolol and the ginger composition is increased synergistically for prophylaxis and/or treatment of irritations of respiratory tract and gastrointestinal tract linings and tissue. The ingestible carrier or coating being present in an amount that causes at least 50 wt-% of the active mixture to be released to the respiratory tract, gastrointestinal tract, or both. The formulation can be in the form of a tablet, a lozenge, a syrup, a capsule, a spray, or a suppository.

The ingestible carrier or coating can provide controlled release of the active mixture. The ingestible carrier or coating is an enteric coating. The ingestible carrier or coating being incorporated in the formulation such that at least 15 wt-% of the active mixture is released to a lower portion of the respiratory tract, wherein the lower portion of the respiratory tract comprises trachea, bronchi, bronchioles and lungs. Alternatively, the ingestible carrier or coating can be incorporated in the formulation such that at least 15 wt-% of the active mixture is released to a lower portion of the gastrointestinal tract, wherein the lower portion of the gastrointestinal tract comprises stomach, small intestine, large intestine, and anus.

The formulation according can be formulated such that, (i) the weight ratio of the ginger composition to bisabolol ranges from 1:10 to 1:100,000; (ii) the combined content of bisabolol and the ginger composition is at least 90 wt-% of the formulation; (iii) the ginger composition comprises 0.001-10 wt-% of the formulation; (iv) the bisabolol comprises 90-99.999 wt-% of the formulation; or a combination thereof.

The formulation can also include natural compositions and mixtures thereof for alleviating the symptoms of the common cold and sinusitis and the various other conditions associated with common cold conditions. These natural compositions and mixtures thereof can include (i) essential oils selected from thyme oil, oil of eucalyptus, oil of wintergreen, peppermint oil, spearmint oil, and combinations thereof; (ii) active substances from essential oils selected from eucalyptol, methyl salicylate, thymol, menthol, and combinations thereof; (iii) natural oral demulcents selected from zinc gluconate, glycene, pectin or a combination thereof; (iv) natural cooling agents selected from menthyl acetate, lactate and combinations thereof; (v) natural warming agents selected from vanillin alcohol ethers, eugenol, cinnamon oil and combinations thereof; (v) additional natural active substances selected from counterirritants, antiseptics, topical analgesics, moisturizers, and combinations thereof; or (vi) combinations thereof.

The formulation can also include synthetic compositions and mixtures thereof for alleviating the symptoms of the common cold and sinusitis and the various other conditions associated with common cold conditions. The synthetic compositions can include (i) inflammation reducers selected from corticosteroid-type steroidal actives, non-steroidal active, and combinations thereof; (ii) antiseptics selected from cetylpyridinium chloride; (iii) topical analgesics or anaesthetics selected from benzocaine, hexylresorcinol and combinations thereof; (iv) antitussives selected from dextromethorphan; and (v) combinations thereof.

The formulation can include a medicament. The medicament can be a medicament for alleviating symptoms of gastroesophageal reflux diseases (esophagitis) and peptic ulcer.

In another embodiment, method of making the formulation is disclose. The method of making the a formulation can include providing an ingestible carrier or coating, and providing an active mixture comprising bisabolol and extracts containing bisabolol and a ginger composition. The active mixture and the ingestible carrier or coating can then be mixed in a manner that at least 15 wt-% of the active mixture to be released to lower portions of a respiratory tract, gastrointestinal tract, or both, when the formulation is introduced to the mouth or endodermal tissue or linings of body cavities. The ginger composition can be (i) a substance mixtures obtainable from an extraction of ginger; (ii) substance mixtures obtainable from a separation of a ginger extract which comprise a compound which is chosen from the group consisting of gingerols, shogaols, gingerdiols, dehydrogingerdiones, paradols and derivatives thereof; (iii) compounds obtainable from a separation of a ginger extract which are chosen from the group consisting of gingerols, shogaols, gingerdiols, dehydrogingerdiones, paradols and derivatives thereof and mixtures thereof, or (iv) a mixture thereof. Finally, the ratio of the bisabolol to the ginger composition in the active mixture is such that a tissue irritation-reducing action of the bisabolol and the ginger composition is increased synergistically for prophylaxis and/or treatment of irritations of respiratory tract and gastrointestinal tract membrane and tissue.

In another embodiment, a method of providing prophylaxis of endodermal tissue irritation and/or for treatment of such irritations and/or inflammations for medical purposes is disclosed. The method can include administering an effective amount of the formulation to a person in need thereof.

### DETAILED DESCRIPTION

The present invention relates to a formulation having inflammation- and/or irritation reducing action on epithelium linings of body cavities of endodermal origin consisting of or comprising: bisabolol or extracts containing bisabolol and a composition or compounds of actives found in ginger, wherein the particular content of bisabolol and the ginger composition or compound is adjusted such that the inflammation-reducing action of these ingredients is increased synergistically.

It also relates to a medicament for treatment of irritations and/or inflammation and the use of a formulation or medicament containing a synergistic mixture of bisabolol and a ginger constituent for prophylaxis of irritation and/or inflammation and/or treatment of irritations and/or inflammations for medical and/or other than medical purposes.

Also disclosed is a process for the preparation of a formulation, a medicament, an OTC drug, either as a liquid or solid dosage form, a glassy or pressed tablet, a capsule, syrup, or a spray, having an inflammation-reducing action. In another embodiment, the invention includes a therapeutic method for prophylaxis and for treatment of irritations or inflammations on endodermal linings, a method for prophylaxis of irritating and/or inflammation action and a method for reducing, eliminating or suppressing the irritating and/or inflammation action of a substance or substance mixture or a kit comprising a formulation having a irritation- and/or inflammation reducing action.

The bisabolol-ginger formulation can be administered either via (i) spraying into the nasal or oral cavity, (ii) oral or nasal inhalation or intermittent positive pressure breathing, (iii) release of the actives in the mouth through dissolution of the carrier with saliva followed by swallowing, or (iv) swallowed directly for controlled release in the upper or lower GI tract.

In the pharmaceutical industry, there is a constant need for agents having an irritation- and/or inflammation reducing action on epithelium and linings of the GI tract and the respiratory tract.

The linings of endodermal origin act as an internal barrier organ, when the human organism is subjected to external influences to a particular extent. Many intrinsic (e.g., genetic predisposition) and extrinsic (e.g., damage to the tissue barrier, irritating or allergy-inducing substances) factors can lead to irritation and/or inflammation of inner linings and epithelium. As used herein, irritation and/or inflammation is to be understood as meaning any change to the linings which induces sensorial malaise in humans or animals and/or is characterized by reddening and/or inflammation. As used herein, the term “sensorial malaise” includes states of pain. As used herein, “irritations” and “inflammations” include phenomenological different linings: irritated or inflamed linings, which may manifest itself as a reddening of the linings.

A large number of active compounds having an irritation- and/or inflammation reducing action are already employed in the technical fields referred to, but alternatives nevertheless continue to be sought. As used herein, an irritation- and/or inflammation reducing action is to be understood as meaning the moderation, reduction, elimination or prevention of irritation and/or inflammation, in particular that of the endodermal symptoms described above. The irritation- and/or inflammation reducing action disclosed herein is based in particular on soothing of the linings, inhibition of inflammation and/or alleviation of reddening. In the search for alternative agents, however, it should be remembered that the substances used must be toxicologically acceptable, stable, and well tolerated by the epithelium, linings and membranes. In particular, in conventional pharmaceutical formulations, such substances should have the lowest possible intrinsic odor and the lowest possible intrinsic color and must be inexpensive to prepare. In accordance with persistent trends towards natural active compounds, novel active compounds of natural, in particular plant, origin are desirable.

The compound (−)-(4S,8R)-alpha-epi-Bisabolol is a natural ingredient of Citrus bergamia RISSO essential oil [(Ohloff, G.; Giersch, W.; Naf, R.; Delay, F.; Helv. Chim. Acta 1986, 69, 698)] and its enantiomer (+)-(4R,8S)-alpha-epi-Bisabolol was isolated from various Abies and Picea species [O'Donnel, G. W.; Sutherland, M. D.; Aust. J. Chem. 1989, 42, 2021], while (+)-(4R,8R)-alpha-Bisabolol is a constitutent of Atalantia monophylla corren oils [O'Donnel, G. W.; Sutherland, M. D., Aust. J. Chem. 1989, 42, 2021 Babin, D.; Fourneron, J. D.; Julia, M.; Tetrahedron 1981, 37 (suppl.] and its enantiomer (−)-(4S,8S)-alpha-Bisabolol is a main constitutent of German Chamomile [Jellinek, J. S.; Parf. Cosm. Aromes 1984, 57, 55]. In addition, (−)-(4S,8S)-alpha-Bisabolol is produced on industrial scale for various applications for cosmetics, flavors and fragrances, for example for protection ointments, lotions, deodorants, because of its anti-inflammatoriy, bacteriostatic und antimycotic properties [Fleischhauer, J.; Beyer, J.; Reinhard, E.; Planta Med. 1990, 56, 456].

Persons skilled in the art have already addressed extensively the skin irritation-reducing properties of bisabolol or extracts containing bisabolol. Similarly, there have been studies of ginger (Zingiber officinale) extract and the substances contained in it, such as gingerols, shogaols, gingerdiols, dehydrogingerdiones and paradols and derivatives thereof.

However, there was no indication hitherto that the mixtures of ginger extract or the compounds contained in this extract (“ginger constituent”) with bisabolol or extracts containing bisabolol (“bisabolol constituent”) have, compared with the components used individually, a significantly improved, synergistic, irritation- and/or inflammation reducing action on epithelium and internal linings of endodermal origin of cavities in the body or that such combinations require lesser amounts of the agents bring about the same inhibitory effect than either single agent alone. As used herein, synergistic action is to be understood as meaning an action which is increased beyond the additive action of the compounds displaying synergy, recorded via the synergy index (SI) value.

Substance combinations in which both components display the synergistically increased action, and also substance combinations in which only one component displays the synergistically increased action, while the other component acts merely as an intensifier (booster), fall under the given definition of the synergy effect. A synergistic combination of active compounds has the advantage that overall less active compound is required to achieve the particular action.

The irritation-reducing action of bisabolol on cutaneous and subcutaneous skin is described in detail (e.g. H. Schilcher, Camomile: Handbook for Doctors, Pharmacists and Other Scientists, Wissenschaftliche Verlagsgesellschaft, Stuttgart, 1987).

The skin irritation-reducing and anti-inflammatory action of ginger (Zingiber officinale) extracts and the substances contained in them, in particular the gingerols, shogaols, gingerdiols, dehydrogingerdiones and paradols and derivatives thereof, is known (E. Tjendraputra et al., Bioorg. Chem. 2001, 29, 156-163; S. D. Jolad et al., Phytochem. 2004, 65, 1937-1954).

The objective of the present invention was therefore to provide a combination of components which also have an improved irritation-and/or inflammation reducing action for linings of the upper and lower GI tract, the upper and lower respiratory tract, or both. This object is achieved by a formulation having a irritation- and/or inflammation reducing action consisting of or comprising:

bisabolol or extracts containing bisabolol and a composition or compound chosen from the group consisting of
a) substance mixtures obtainable from an extraction of ginger,
b) substance mixtures obtainable from a separation of a ginger extract which comprise a compound which is chosen from the group consisting of gingerols, shogaols, gingerdiols, dehydrogingerdiones, paradols and derivatives thereof and
c) compounds obtainable from a separation of a ginger extract which are chosen from the group consisting of gingerols, shogaols, gingerdiols, dehydrogingerdiones, paradols and derivatives thereof and mixtures thereof,

wherein the particular content of bisabolol and of the ginger composition or compound in the formulation is adjusted such that the irritation-reducing action of these compounds is increased synergistically when administered to treat irritations and inflammations of endodermal tissue.

Bisabolol is a very effective 1L-1alpha (1L-1α) and LTB4 inhibitor, but not as a PGE2 inhibitor. Ginger extracts contain very effective PGE2 inhibitors, which are useful to treat acute inflammation. Gingerol has been shown to inhibit activation of TNF-α and COX-2 expression and to inhibit PGE2 production by HL-60 cells; however, it did not have an effect on IL-1α inhibition. Surprisingly, the combined action of bisabolol and gingerol via IL-1α inhibition, LTB4 and PGE2 inhibition seems to be the principle responsible for the synergistic properties for reducing inflammation and irritation of endodermal tissues.

It was particularly surprising that the formulation according to the invention shows a highly synergistic activity when a preparation is ingested or administered internally by other means to irritated or inflamed linings of inner cavities. The synergistic formulations are significantly superior to individually dosed bisabolol or individually dosed ginger extract at the same concentration.

It has also been found that this synergistic activity of the reduction in irritation and/or inflammation is not limited solely to acceleration of the subsidence (“repair”) of the inflammation and/or reddening of the linings compared with untreated linings. Rather, the formulation according to the invention also has a highly synergistic action in reducing the development of inflammations (“protection”), e.g., due to the above mentioned factors.

On the basis of the particularly significant increase in the action of its constituents, the formulation according to the invention is particularly suitable for reducing irritation and inflammation, in particular for soothing the linings and/or inhibiting inflammation and/or reducing reddening, even at a low dosage of the bisabolol constituent-ginger constituent formulations according to the invention.

For preparation of an effective synergistic mixture according to the invention comprising a ginger constituent and bisabolol constituent which causes a particularly efficient reduction irritation, it is sufficient to mix bisabolol constituent with a small amount of ginger constituent. In this context, the ginger constituent is:

a) a ginger (Zingiber officinale) extract and/or

b) a substance mixture of the same composition as a ginger extract and/or

c) a substance mixture which is prepared from a separation of a ginger extract and comprises a compound which is chosen from the group consisting of gingerols, shogaols, gingerdiols, dehydrogingerdiones, paradols and derivatives thereof and/or

d) a compound which is prepared from a separation of a ginger extract and is chosen from the group consisting of gingerols, shogaols, gingerdiols, dehydrogingerdiones, paradols and derivatives thereof and/or

e) a substance mixture which is of the same composition as a substance mixture prepared from a separation of a ginger extract and comprises a compound which is chosen from the group consisting of gingerols, shogaols, gingerdiols, dehydrogingerdiones, paradols and derivatives thereof and/or

f) a compound such as can be prepared from a separation of a ginger extract, which is chosen from the group consisting of gingerols, shogaols, gingerdiols, dehydrogingerdiones, paradols and derivatives thereof, and the bisabolol constituent is: bisabolol.

The ginger constituent is very particularly preferably ginger extract, in particular ginger (Zingiber officinale) extract.

Preferably, the weight ratio of the ginger constituent, preferably ginger extract, to the bisabolol constituent is in the range of from 1:100,000 to 1:10, preferably in the range of from 1:10,000 to 1:20, and particularly preferably in the range of from 1:1000 to 1:50. Preferably, the synergistic mixture of the ginger constituent (preferably ginger extract) and the bisabolol constituent according to the invention comprises at least 90 wt. %, preferably at least 95 wt. %, particularly preferably at least 98 wt. %, based on the total weight of the synergistic mixture according to the invention.

Preferably, the amount of (preferably ginger extract) (preferably ginger extract) is in the range of 0.001-10 wt. %, particularly preferably in the range of 0.01-5 wt. %, in particular in the range of 0.1-2.0 wt. %, based on the total weight of the synergistic mixture according to the invention and/or the content of the bisabolol constituent is preferably 90-99.999 wt. %, particularly preferably 95-99.99 wt. %, in particular 98-99.9 wt. %, based on the total weight of the synergistic mixture according to the invention.

The total amount of the ginger constituent (preferably ginger extract) and the bisabolol constituent used in ready-to-use treatment formulations according to the invention is preferably 0.001-5.0 wt. %, particularly preferably 0.01-1.0 wt. %, in particular 0.01-0.25 wt. %, based on the total weight of the treatment formulation. In the connection of this text, “ready-to-use” is to be understood as meaning that the treatment formulation is intended for coming into contact with the tissue in an unchanged form.

The bisabolol constituent used in the context of the present invention can be of natural or synthetic origin, and is preferably “alpha-bisabolol”. As used herein, the term “alpha-bisabolol” includes (+)-alpha-bisabolol, (−)-alpha-bisabolol, (+)-epi-alpha-bisabolol and (−)-epi-alpha-bisabolol and combinations thereof. In particular, the term “alpha-bisabolol” can include racemic mixtures of (±)-alpha-bisabolol and/or (±)-epialpha-bisabolol. Preferably, the bisabolol constituent used is synthetically prepared or natural (−)-alpha-bisabolol and/or synthetic mixed-isomer alpha-bisabolol. If natural (−)-alpha-bisabolol is used, this can also be employed as a constituent of an essential oil or of a plant extract or of a fraction thereof, for example as a constituent of (fractions of) oil or extracts of chamomile or of Vanillosmopsis (in particular Vanillosmopsis erythropappa or Vanillosmopsis arborea). Synthetic alpha-bisabolol can be obtained, for example, under the name DRAGOSANTOL from Symrise.

The ginger constituent employed for preparation of the formulation according to the invention is, in the case of ginger extract (also as a precursor), preferably an extract of the fresh or dried ginger root which is prepared by extraction with methanol, ethanol, iso-propanol, acetone, ethyl acetate, carbon dioxide (CO₂), hexane, methylene chloride, chloroform or other solvents or solvent mixtures of comparable polarity. The extracts are characterized by the presence of active endodermal tissue irritation-reducing amounts of constituents including, but not limited to, gingerols, shogaols, gingerdiols, dehydrogingerdiones and/or paradols. Essential ginger oils, obtained by steam distillation, are not suitable as a constituent in the context of the synergistic formulations according to the invention due to the absence of active endodermal tissue irritation-reducing amounts of constituents such as gingerols, shogaols, gingerdiols, dehydrogingerdiones and/or paradols.

For the separation of a ginger extract for the preparation of the ginger constituent, it is not difficult for the person skilled in the art to choose suitable separation methods known from the prior art. The person skilled in the art can also choose suitable processes from the prior art for the preparation of “synthetic” ginger extracts, i.e., mixtures that correspond to ginger extracts in their composition but have not been obtained by extraction from ginger. The same also applies to substance mixtures and compounds which are obtainable from a separation of a ginger extract but have been prepared in a manner other than by separation of a ginger extract.

A formulation according to the invention having an irritation- and/or inflammation reducing action wherein the contents of both the ginger constituent and the bisabolol constituent have an irritation- and/or inflammation reducing action is preferred. The advantage of the preferred formulation according to the invention lies in the fact that such formulations reduce irritations and/or inflammation of the epithelium in the upper and lower gastrointestinal tract, the upper and lower respiratory tract, or both, particularly effectively.

The invention also provides a medicament for treatment of irritations and inflammations, comprising or consisting of a formulation according to the invention having an irritation- and/or inflammation reducing action. Such a medicament can be employed in the field of human and veterinary medicine against a large number of diseases and inflammation processes of inner linings of endodermal origin in the upper gastrointestinal tract, including, but not limited to, inflammation of the esophagus and the stomach. Such medicaments can also be used for treating regions of the upper and lower respiratory tract and lower gastrointestinal tract.

A formulation according to the invention can be further processed into solid formulation by optionally adding a pharmaceutically and/or food acceptable solid carrier or excipient to the formulation and then drying the mixture by suitable processes. In this context, such a solid which is not toxic to the organisms being treated with the formulation.

The formulation according to the invention can also be further processed to a diluted formulation in liquid form by optionally adding a pharmaceutically acceptable or food-grade solvent, e.g., neutral oil, plant oils, ethanol, 1,2-propylene glycol, water, and mixtures thereof. These formulations according to the invention can optionally be prepared with the addition of a solubilization agent, preservative or antioxidant, such as, EXTRAPON Ginger obtainable from Symrise.

The formulation according to the invention, whether a liquid or a solid formulation, can also be further processed by encapsulation. According to the invention, the formulation according to the invention and/or the liquid or solid formulation comprising the formulation can be encapsulated with a solid shell material. The solid material can be a material selected from starches, degraded or chemically or physically modified starches (in particular dextrins and maltodextrins, glucose syrup, water-soluble fibers), gelatins, wax materials, liposomes, gum arabic, agar-agar, ghatti gum, gellan gum, modified and non-modified celluloses, pullulan, curdlan, carrageenans, algic acid, alginates, pectin, pectinates, inulin, xanthan gum and mixtures thereof. The solid shell material can be an enteric coating. The formulation according to the invention could be added to a solid carrier or an aqueous solution of a carrier, which is processed by other means, such as melt extrusion, film casting, spray drying, agglomeration, granulation, spheronization, coating, etc.

The ingestible carrier or coating can cause at least 50 wt-% of the active mixture to be released to the respiratory tract, gastrointestinal tract, or both. The ingestible carrier or coating can cause at least 75 wt-%, at least 90 wt-%, at least 95 wt-% or at least 99 wt-% of the active mixture to be released to the respiratory tract, the gastrointestinal tract, or both.

The formulation can be formulated such that the ingestible carrier or coating provides a controlled release of the active mixture. For example, at least 15 wt-% of the active mixture can be released to a lower portion of the respiratory tract, a lower portion of the gastrointestinal tract, or a combination thereof. The formulation can be formulated such that at least 33 wt-%, at least 50 wt-%, at least 75 wt-% or at least 90 wt-% of the active mixture can be released to a lower portion of the respiratory tract, a lower portion of the gastrointestinal tract, a combination thereof.

As used herein, the lower portion of the respiratory tract refers to the trachea, bronchi, bronchioles and lungs. As used herein, the lower portion of the gastrointestinal tract refers to the stomach, small intestine, large intestine, and anus.

A further aspect of the present invention relates to formulations according to the invention in the form of prescription and over-the-counter (OTC) drugs or medicines.

OTC drugs or medicines can be cough medicines with antitussives or expectorants (cough drops, lozenges or syrups), stomach remedies (antacids), or nasal sprays.

Tablets or soft and hard gelatin-type capsules with or without enteric coatings could be used for administering the formulations according to the invention for treatment of gastrointestinal irritation and inflammation.

Formulations according to the invention, can additionally comprise one or more aroma and/or flavoring substances, such as essential oils and extracts, tinctures and balsams, such as, for example, anisole, basil oil, bergamot oil, bitter almond oil, camphor oil, citronella oil, lemon oil; Eucalyptus citriodora oil, eucalyptus oil, fennel oil, grapefruit oil, ginger oil, chamomile oil, spearmint oil, caraway oil, lime oil, mandarin oil, nutmeg oil (in particular nutmeg blossom oil =maces oil, mace oil), myrrh oil, clove oil, clove blossom oil, orange oil, oregano oil, parsley (seed) oil, peppermint oil, rosemary oil, sage oil (clary sage, Dalmatian or Spanish sage oil), star aniseed oil, thyme oil, vanilla extract, juniper oil (in particular juniper berry oil), wintergreen oil, cinnamon leaf oil; cinnamon bark oil, and fractions thereof, or constituents isolated therefrom.

It is of particular advantage if the formulations according to the invention comprise at least one aroma substance, preferably 2 to 10 or more aroma substances, chosen from the following group: menthol (preferably L-menthol and/or racemic menthol), anethole, anisole, anisaldehyde, anisyl alcohol, neomenthol, eucalypt (1,8-cineol), menthone (preferably L-menthone), isomenthone (preferably D-isomenthone), isopulegol, menthyl acetate (preferably L-menthyl acetate), menthyl propionate, carvone (preferably (−)-carvone, optionally as a constituent of a spearmint oil), methyl salicylate (optionally as a constituent of a wintergreen oil), eugenol acetate, isoeugenol methyl ether, beta-homocyclocitral, eugenol, isobutyraldehyde, 3-octanol, dimethyl sulfide, hexanol, hexanal, trans-2-hexenal, cis-3-hexenol, 4-terpineol, piperitone, linalool, 8-ocimenyl acetate, isoamyl alcohol, isovaleraldehyde, alphapinene, beta-pinene, limonene (preferably D-limonene, optionally as a constituent of an essential oil), piperitone, trans-sabinene hydrate, menthofuran, caryophyllene, germacrene D, cinnamaldehyde, mint lactone, thymol, gamma-octalactone, gamma-nonalactone, gamma-decalactone, (1,3E,5Z)-undecatriene, 2-butanone, ethyl formate, 3-octyl acetate, isoamyl isovalerate, cis and trans-carvyl acetate, p-cymol, damascenone, damascone, cis-rose oxide, trans-rose oxide, fenchol, acetaldehyde diethyl acetal, 1-ethoxyethyl acetate, cis-5 4-heptenal, cisjasmone, methyl dihydrojasmonate, 2′-hydroxypropiophenone, menthyl methyl ether, myrtenyl acetate, 2-phenylethyl alcohol, 2-phenylethyl isobutyrate, 2-phenylethyl isovalerate, geraniol, nerol and viridiflorol.

The formulations according to the invention advantageously comprise cooling agents. Preferred cooling active compounds for use in formulations according to the invention for lozenges, cough drops, and capsules are listed in the following. The person skilled in the art can supplement the following list with a large number of further cooling active compounds and the cooling active compounds can also be employed in combination with one another. Preferably, the formulations according to the invention comprise at least one cooling active compound, preferably two or more cooling active compounds, chosen from:

Menthone glycerol acetal (trade name: Frescolat®, Symrise GmbH & Co. KG, Germany), menthyl lactate (trade name: Frescolat®ML, Symrise GmbH & Co. KG, Germany, menthyl lactate is preferably 1-menthyl lactate, in particular 1-menthyl I-lactate), substituted menthyl-3-carboxylic acid amides (e.g. menthyl-3-carboxylic acid N-ethylamide, also known as WS-3), 2-isopropyl-N-2,3-trimethylbutanamide (also known as WS-23), substituted cyclohexanecarboxylic acid amides, 3-menthoxypropane-1,2-diol, 2-hydroxyethyl menthyl carbonate, 2-hydroxypropyl menthyl carbonate, N-acetylglycine menthyl ester, isopulegol, menthyl hydroxycarboxylic acid esters (e.g. menthyl 3-hydroxybutyrate), monomenthyl succinate, 2-mercaptocyclodecanone, menthyl 2-pyrrolidin-5-onecarboxylate, 2,3-dihydroxy-p-menthane, 3,3,5-trimethylcyclohexanone glycerol ketal, 3-menthyl 3,6-di- and -trioxaalkanoates, 3-menthyl methoxyacetate and icilin.

Particularly preferred cooling active compounds are: menthone glycerol acetal (trade name: Frescolat®MGA), menthyl lactate (preferably 1-menthyl lactate, in particular 1-menthyl I-lactate, trade name: Frescolat®ML), substituted menthyl-3-carboxylic acid amides (e.g. menthyl-3-carboxylic acid N-ethylamide), 2-isopropyl-N-2,3-trimethylbutanamide, 3-menthoxypropane-1,2-diol, 2-hydroxyethyl menthyl carbonate, 2-hydroxypropyl menthyl carbonate, isopulegol and monomenthyl succinate, and mixtures thereof, i.e. Optacool® (Symrise, Germany).

Formulations according to the invention which comprise I-menthol and at least one, particularly preferably at least two cooling substances are preferred according to the invention.

Preferably, a formulation according to the invention comprises a mixture of flavoring and/or aroma substances which imparts to a formulation according to the invention an overall herbal (herb-like), minty, cinnamon-like, clove-like, wintergreen and/or fruity character.

Physiological warming agents can also be used. Exemplary physiological warming agents include vanillyl alcohol n-butyl ether, vanillyl alcohol, n-propylether, vanillyl alcohol, isopropyl ether, vanillyl alcohol isobutyl ether, vanillyl alcohol, n-amino ether, vanillyl alcohol isoamyl ether, vanillyl alcohol n-hexyl ether, vanillyl alcohol methyl ether, vanillyl alcohol ethyl ether, gingerol, shogaol, paradol, zingerone, capsaicin, dihydrocapsaicin, nordihydrocapsaicin, homocapsaicin, homodihydrocapsaicin, ethanol, iso-propyl alcohol, iso-amylalcohol, benzyl alcohol, chloroform, eugenol, cinnamon oil, cinnamic aldehyde, phosphate derivatives thereof, and mixtures thereof. The phosphate derivatives can include those described in WO 97102273. A commercial example of a suitable warming agent for use herein is Optaheat® (Symrise, Germany). The balance of the warming composition may be made up of a suitable appropriate carrier, such as water, propylene glycol or a bulk sweetener, described in more detail below. The warming composition can further comprise a cooling agent as described herein provided that the predominant effect is one of warming.

As a salivating or moisturizing agent in the preparation of a medicament for soothing irritated oral tissues the salivating agent comprises trans-pelletorin or Optaflow® (Symrise, Germany), as described in US 2004/0241312A and EP 1520850A2, or other actives, such as Jambu and Spilanthrol. They modulate oral and nasal secretion, providing long-lasting moisturization of the oral and nasal tissue due to its lipophilicity by inducing salivation, in comparison with organic acids such as citric acid.

The pharmaceutical formulations according to the invention can comprise cosmetic auxiliary substances and additives such as are conventionally used in such formulations including preservatives, bactericides, fungicides, virucides, cooling active compounds, plant extracts, plant parts, anti-inflammatory active compounds, substances which accelerate wound healing (e.g. chitin or chitosan and derivatives thereof), film-forming substances (e.g. polyvinylpyrrolidones or chitosan or derivatives thereof, and hydrocolloidal products derived from algae, such as alginates), antioxidants, vitamins, 2-hydroxycarboxylic acids (e.g. citric acid, malic acid, L-, D- or dl-lactic acid), softening, moisturizing and/or humectant substances, fats, oils, saturated fatty acids, mono- or polyunsaturated fatty acids, a-hydroxy acids, polyhydroxy-fatty acids or derivatives thereof, thickening agents, surface-active substances and emulsifiers.

The formulations according to the invention can also preferably comprise further irritation-or inflammation-reducing active compounds. In this case, all the irritation-reducing active compounds which are suitable or useful for medical uses and ingestion can be used in this respect. Steroidal anti-inflammatory substances of the corticosteroid type are advantageously employed in this regard. Exemplary corticosteroids include, for example, hydrocortisone, hydrocortisone derivatives, such as hydrocortisone 17-butyrate, dexamethasone, dexamethasone phosphate, methylprednisolone or cortisone, it being possible for the list to be extended to additional steroidal anti-inflammatories.

Non-steroidal anti-inflammatories can also be employed. There are to be mentioned here by way of example oxicams, such as piroxicam or tenoxicam; salicylates, such as aspirin, fendosal acetic acid derivatives, such as diclofenac, fenclofenac, indomethacin, sulindac, tolmetin or clindanac; fenamates, such as mefenamic, meclofenamic, flufenamic or niflumic; propionic acid derivatives, such as ibuprofen, naproxen or benoxaprofen; or pyrazoles, such as phenylbutazone, oxyphenylbutazone, febrazone or azapropazone.

The formulations according to the invention can also preferably comprise further irritation-or inflammation-reducing active compounds and corresponding actives for alleviating symptoms related to gastro-esophagal reflux disease, peptic ulcers, etc. In these embodiments, all the irritation-reducing active compounds which are suitable or useful for medical uses and ingestion can be used in this respect. Antacids such as aluminum and/or magnesium hydroxids, calcium carbonate, sodium bicarbonate, hydrotalcite, bismuth subsalicylate, magaldrate plus simethicone. Steroidal anti-inflammatory substances of the corticosteroid type can advantageously be employed for this, such as hydrocortisone, hydrocortisone derivatives, such as hydrocortisone 17-butyrate, dexamethasone, dexamethasone phosphate, methylprednisolone or cortisone, it being possible for the list to be extended by addition of further steroidal anti-inflammatories. Non-steroidal anti-inflammatory substances of the cyclooxygenase-1 and -2 type inhibitor, such as celecoxib or rofecoxib, can also be employed advantageously. The formulation according to the invention can also comprise proton pump inhibitors (PPI), such as omeprazole or esomeprazole, and Gastric H₂ receptor blockers, such as ranitidine or famotidine.

Alternatively, natural anti-inflammatory substances or reddening-alleviating substances can be employed. Plant extracts, specific highly active plant extract fractions and highly pure active substances isolated from plant extracts, can be employed. Exemplary extracts, fractions and active substances from plants include those from aloe vera, Commiphora species, Rubia species, willow, rosebay willow-herb, oats, calendula, arnica, St. John's wort, honeysuckle, rosemary, Melissa, Passiflora incarnata, witch hazel, Pueraria, Dianthus or Echinacea, as well as pure substances, such as, inter alia, apigenin, apigenin 7-glucoside, rosemary acid, boswellic acid, phytosterols, glycyrrhizic acid, glabridin, licochalcone A and anthranilic acid amides, such as, in particular, avenanthramides or dianthramides, are particularly preferred.

The amount of anti-irritants (one or more compounds) in the formulations is preferably 0.0001 to 20 wt. %, particularly preferably 0.0001 to 10 wt. %, in particular 0.001 to 5 wt. %, based on the total weight of the formulation.

The formulations according to the invention can also comprise antioxidants, it being possible for all the antioxidants which are suitable or useful for medical uses to be used.

The amount of antioxidants (one or more compounds) in the formulations according to the invention is preferably 0.01 to 20 wt. %, particularly preferably 0.05 to 10 wt. %, in particular 0.2-5 wt. %, based on the total weight of the formulation.

Formulations according to the invention can also be employed together with osmolytes. Exemplary osmolytes can include substances such as sugar alcohols (myo-inositol, mannitol, sorbitol), quaternary amines, such as taurine, choline, betaine, betaine-glycine and ectoin, diglycerol phosphate, phosphorylcholine, glycerophosphorylcholines, amino acids, such as glutamine, glycine, alanine, glutamate, aspartate or proline, phosphatidylcholine, phosphatidylinositol and inorganic phosphates, as well as polymers of the compounds mentioned, such as proteins, peptides, poly-amino acids and polyols.

Formulations according to the invention can advantageously also comprise vitamins and vitamin precursors and combinations thereof. Vitamins and vitamin precursors which may be mentioned by way of example are: vitamin A (retinol) and its derivatives, vitamin B1 (thiamine) and its salts, vitamin B12 (cobalamin), vitamin B2 (vitamin G, riboflavin) and its derivatives, vitamin B3 and its derivatives, vitamin B4 (adenine) and its derivatives, provitamin B5, vitamin B5 (pantothenic acid) and its derivatives, vitamin B6 (pyridoxol, pyroxidal, pyridoxamine) and its derivatives, vitamin C (ascorbic acid) and its derivatives, provitamin D, vitamin D (calciol) and its derivatives (e.g. vitamin D2, vitamin D3), vitamin E (D-alpha-tocopherol) and its derivatives, vitamin F (essential fatty acids, linolenic acid and linoleic acid) and its derivatives (e.g. vitamin F ethyl ester, vitamin F glyceryl ester), vitamin H (vitamin B7, biotin), vitamin K1 (phylloquinone, phytonadione) and vitamin K3 (menadione, menaquinone).

Formulations according to the invention can likewise comprise one or more further plant extracts, which are conventionally prepared by extraction of the whole plant, but in individual cases also exclusively from blossom and/or leaves, wood, bark or roots of the plant. Extracts which are advantageous can include aloe, algae, apple, apricot, arnica, avocado, pear, stinging nettle, blackberry, calendula, ivy, hibiscus, oak bark, strawberry, spruce, honeysuckle, barley, ginkgo, ginseng, pomegranate, grapefruit, cucumber, oats, witch hazel, restharrow, henna, raspberry, elder, honeybush, hops, coltsfoot, kiwi, burdock, coconut, lavender, lime, linden, mallow, almond, mango, box holly, Melissa, olive, orange, peppermint, Pueraria, wild thyme, rooibos, rose, rosemary, horse chestnut, sage, sandalwood, yarrow, horsetail, Sophora, licorice, dead nettle, tea (green, white, black), thyme, grape, juniper, willow, rose-bay willow-herb, hawthorn, wheat, lady's smock, cinnamon, lemon and lemongrass. In this context, the extracts from aloe Vera, algae, arnica, stinging nettle, calendula, witch hazel, linden, ginseng, cucumber, rosemary and sage are particularly preferred. Mixtures of two or more plant extracts can also be employed.

Extraction agents which can be used for the preparation of the plant extracts mentioned include water, alcohols and mixtures thereof. In this context, the alcohols can be lower alcohols, such as ethanol and isopropanol, and also polyhydric alcohols, such as ethylene glycol, propylene glycol and butylenes glycol, are preferred, and in particular both as the sole extraction agent and in mixtures with water. The plant extracts can be employed both in the pure and in the diluted form.

In various cases it may also be advantageous to employ formulations according to the invention in combination with substances which are chiefly employed for inhibition of the growth of undesirable microorganisms on or in animal organisms. In this respect, in addition to conventional preservatives and antibiotics, further active compounds that can be included in the synergistic formulations include chitosan, famesol, glycerol monolaurate, polyglycerol esters, such as polyglyceryl 3-caprylates, fatty acid monoesters of sugar, such as sucrose monolaurate, and combinations thereof.

Formulations according to the invention can also comprise preservatives. Preservatives which are preferably chosen here are those such as benzoic acid and its esters and salts, propionic acid and its esters and salts, salicylic acid and its esters and salts, 2,4-hexadienoic acid (sorbic acid) and its esters and salts.

The invention also provides the use of a formulation according to the invention or of a medicament according to the invention for prophylaxis and/or for treatment of irritations and/or inflammations for medical and/or other than medical purposes. The invention likewise provides the use of a formulation according to the invention or of a medicament according to the invention for the preparation of a medicament for treatment of irritations and inflammation.

The invention furthermore provides the use of a formulation according to the invention or of a medicament according to the invention for the preparation of a pharmaceutical formulation.

The invention also provides the use of a formulation according to the invention or of a medicament according to the invention for reducing, eliminating or suppressing the endodermal tissue-irritating action of a substance or substance mixture.

The advantage of the uses according to the invention lies in particular in that due to the synergistic effect of the components contained in the formulation according to the invention or in the medicament according to the invention in respect of its irritation- and/or inflammation reducing action, relatively low active combinations are sufficient.

This reduces the probability of a renewed allergic reaction, and can include cost advantages and contribute towards protecting the environment.

The invention also provides a process for the preparation of a formulation according to the invention or of a medicament according to the invention, with the following steps:

provision of bisabolol,

provision of a ginger composition or compound chosen from the group consisting of

substance mixtures obtainable from an extraction of ginger,

substance mixtures obtainable from a separation of a ginger extract which comprise a compound which is chosen from the group consisting of gingerols, shogaols, gingerdiols, dehydrogingerdiones, paradols and derivatives thereof,

compounds obtainable from a separation of a ginger extract which are chosen from the group consisting of gingerols, shogaols, gingerdiols, dehydrogingerdiones, paradols and derivatives thereof,

mixing of the components provided, so that the particular content of bisabolol and that of the ginger composition or compound in the mixture is adjusted, such that the irritation- and/or inflammation reducing action of these contents is increased synergistically.

The invention likewise provides a therapeutic method for prophylaxis of irritation and inflammation, with the following steps:

provision of a formulation according to the invention or of a medicament according to the invention and

application of the formulation or of the medicament to non-irritated tissue in an active amount.

The invention furthermore provides a therapeutic method for treatment of irritation and/or inflammations, with the following steps:

provision of a formulation according to the invention or of a medicament according to the invention and application of the formulation or of the medicament to irritated and/or inflamed linings and epithelium of endodermal origin in an active amount.

The invention furthermore provides a method for prophylaxis of the irritating and inflammation action or for reducing, eliminating or suppressing the irritating and inflammation action of a substance or substance mixture, with the following steps:

provision of a substance or substance mixture having a endodermal tissue-irritating action, provision of bisabolol, provision of a ginger composition or compound chosen from the group consisting of substance mixtures obtainable from an extraction of ginger, substance mixtures obtainable from a separation of a ginger extract which comprise a compound which is chosen from the group consisting of gingerols, shogaols, gingerdiols, dehydrogingerdiones, paradols and derivatives thereof and compounds obtainable from a separation of a ginger extract which are chosen from the group consisting of gingerols, shogaols, gingerdiols, dehydrogingerdiones, paradols and derivatives thereof

bringing together of the last two components provided with the substance or substance mixture having a endodermal tissue-irritating action, so that the irritating and/or inflammation action is reduced, eliminated or suppressed and a formulation according to the invention or a medicament according to the invention is formed.

One advantage of the therapeutic method is that the irritating and/or inflammation action of compounds or compound mixtures can be moderated in this way to the extent that they are accessible for uses for which they were hitherto not available. On the basis of the method according to the invention mentioned last, higher concentrations of irritation and inflammation compounds and mixtures can also be employed in uses where there is the possibility of contact with the linings and epithelium of endodermal origin. In this context, it is particularly preferable if, on the basis of the method according to the inventive therapeutic method, the lining-irritating action of the irritating or inflammation causing compound is eliminated completely or is suppressed completely (i.e. it no longer has an effect) by the mixture of bisabolol constitutent and the ginger constituent.

For the methods and uses mentioned, the ratios of amounts or contents of the ginger constituent and the bisabolol constituentand of the formulation according to the invention which are described above as preferred likewise apply.

Preferred embodiments and further aspects of the present invention emerge from the attached patent claims and the following examples, the examples not being intended to limit the invention.

1. A formulation having a irritation-reducing action, an inflammation reducing action, or both, comprising:
a. an ingestible carrier or coating; and
b. an active mixture comprising bisabolol or extracts containing bisabolol and a ginger composition selected from the group consisting of:
i. substance mixtures obtainable from an extraction of ginger,
ii. substance mixtures obtainable from a separation of a ginger extract which comprise a compound which is chosen from the group consisting of gingerols, shogaols, gingerdiols, dehydrogingerdiones, paradols and derivatives thereof,
iii. compounds obtainable from a separation of a ginger extract which are chosen from the group consisting of gingerols, shogaols, gingerdiols, dehydrogingerdiones, paradols and derivatives thereof and mixtures thereof, and
iv. mixtures thereof,
wherein the ratio of the bisabolol to the ginger composition in the active mixture is such that an irritation reducing action, an inflammation reducing action, or both, of the bisabolol and the ginger composition is increased synergistically for prophylaxis and/or treatment of irritations of respiratory tract and gastrointestinal tract linings and tissue; and
wherein the ingestible carrier or coating causes at least 50 wt-% of the active mixture to be released to the respiratory tract, gastrointestinal tract, or both.

2. The formulation of claim 1, wherein the formulation is a tablet, a lozenge, a syrup, a capsule, a spray, or a suppository.

3. The formulation of claim 1, wherein the ingestible carrier or coating provides a controlled release of the active mixture.

4. The formulation of claim 1, wherein the ingestible carrier or coating is an enteric coating.

5. The formulation of claim 1, wherein at least 15 wt-% of the active mixture is released to a lower portion of the respiratory tract, wherein the lower portion of the respiratory tract comprises trachea, bronchi, bronchioles and lungs.

6. The formulation of claim 1, wherein at least 15 wt-% of the active mixture is released to a lower portion of the gastrointestinal tract, wherein the lower portion of the gastrointestinal tract comprises stomach, small intestine, large intestine, and anus.

7. The formulation according to claim 1, wherein:
i. the weight ratio of the ginger composition to bisabolol ranges from 1:10 to 1:100,000;
ii. the combined content of bisabolol and the ginger composition is at least 90 wt-% of the formulation;
iii. the ginger composition comprises 0.001-10 wt-% of the formulation;
iv. the bisabolol comprises 90-99.999 wt-% of the formulation; or
v. a combination of (i)-(iv).

8. The formulation according to claim 1, further comprising natural compositions and mixtures thereof for alleviating the symptoms of the common cold and sinusitis and the various other conditions associated with common cold conditions, comprising:
essential oils selected from thyme oil, oil of eucalyptus, oil of wintergreen, peppermint oil, spearmint oil, and combinations thereof;
active substances from essential oils selected from eucalyptol, methyl salicylate, thymol, menthol, and combinations thereof;
natural oral demulcents selected from zinc gluconate, glycene, pectin or a combination thereof;
natural cooling agents selected from menthyl acetate, lactate and combinations thereof;
natural warming agents selected from vanillin alcohol ethers, eugenol, cinnamon oil and combinations thereof;
additional natural active substances selected from counterirritants, antiseptics, topical analgesics, moisturizers, and combinations thereof; or combinations thereof.

9. The formulation according to claim 1, further comprising synthetic compositions and mixtures thereof for alleviating the symptoms of the common cold and sinusitis and the various other conditions associated with common cold conditions, comprising:
inflammation reducers selected from corticosteroid-type steroidal actives, non-steroidal active, and combinations thereof;
antiseptics selected from cetylpyridinium chloride;
topical analgesics or anaesthetics selected from benzocaine, hexylresorcinol and combinations thereof;
antitussives selected from dextromethorphan; or combinations thereof.

10. The formulation according to claim 1, further comprising medicaments for alleviating symptoms of gastroesophageal reflux diseases (esophagitis) and peptic ulcer.

11. A method of making a formulation according to claim 1, comprising:
a. providing an ingestible carrier or coating;
b. providing an active mixture comprising bisabolol and extracts containing bisabolol and a ginger composition selected from the group consisting of:
1. substance mixtures obtainable from an extraction of ginger,
2. substance mixtures obtainable from a separation of a ginger extract which comprise a compound which is chosen from the group consisting of gingerols, shogaols, gingerdiols, dehydrogingerdiones, paradols and derivatives thereof,
3. compounds obtainable from a separation of a ginger extract which are chosen from the group consisting of gingerols, shogaols, gingerdiols, dehydrogingerdiones, paradols and derivatives thereof and mixtures thereof, and
4. mixtures thereof,
c. wherein the ratio of the bisabolol to the ginger composition in the active mixture is such that a tissue irritation-reducing action of the bisabolol and the ginger composition is increased synergistically for prophylaxis and/or treatment of irritations of respiratory tract and gastrointestinal tract membrane and tissue; and
d. combining the active mixture and the ingestible carrier or coating in a manner that at least 15 wt-% of the active mixture to be released to lower portions of a respiratory tract, gastrointestinal tract, or both, when the formulation is introduced to the mouth or endodermal tissue or linings of body cavities.

12. A method of providing prophylaxis of endodermal tissue irritation and/or for treatment of such irritations and/or inflammations for medical purposes, comprising administering an effective amount of a formulation to a person in need thereof, wherein the formulation comprises an active mixture comprising bisabolol and a ginger composition selected from the group consisting of:
a, substance mixtures obtainable from an extraction of ginger,
b. substance mixtures obtainable from a separation of a ginger extract which comprise a compound which is chosen from the group consisting of gingerols, shogaols, gingerdiols, dehydrogingerdiones, paradols and derivatives thereof,
c. compounds obtainable from a separation of a ginger extract which are chosen from the group consisting of gingerols, shogaols, gingerdiols, dehydrogingerdiones, paradols and derivatives thereof and mixtures thereof, and
d. mixtures thereof;
wherein the ratio of the bisabolol to the ginger composition in the active mixture is such that a endodermal tissue irritation reducing action, inflammation reducing action, or both, of the bisabolol and the ginger composition is increased synergistically for prophylaxis and/or treatment of irritations of respiratory tract and gastrointestinal tract linings and tissue; and
wherein at least 15 wt-% of the active mixture to be released to lower portions of a respiratory tract, gastrointestinal tract, or both.

13. The method of claim 12, wherein the formulation further comprises medicaments for alleviating the symptoms of gastroesophageal reflux diseases (esophagitis), peptic ulcer, or both.

14. The method of claim 12, wherein the formulation further comprises synthetic compositions and mixtures thereof for alleviating the symptoms of the common cold and sinusitis and the various other conditions associated with common cold conditions, comprising:
inflammation reducers selected from corticosteroid-type steroidal actives, non-steroidal active, and combinations thereof;
antiseptics selected from cetylpyridinium chloride;
topical analgesics or anaesthetics selected from benzocaine, hexylresorcinol and combinations thereof;
antitussives selected from dextromethorphan; and
combinations thereof.

15. The method of claim 12, wherein the formulation further comprises a tissue-irritation reducing material, wherein the active mixture eliminates or suppresses the tissue-irritating action of the tissue-irritating material.

16. The method of claim 12, wherein the formulation is a tablet, a capsule, or a suppository.

17. The method of claim 12, wherein the ingestible carrier or coating provides a controlled release of the active mixture.

18. The method of claim 12, wherein the ingestible carrier or coating is an enteric coating.

19. The method of claim 12, wherein at least 15 wt-% of the active mixture is released to a lower portion of the respiratory tract, wherein the lower portion of the respiratory tract comprises trachea, bronchi, bronchioles and lungs.

20. The method of claim 12, wherein at least 15 wt-% of the active mixture is released to a lower portion of the gastrointestinal tract, wherein the lower portion of the gastrointestinal tract comprises small intestine, large intestine, and anus.

21. The method according to claim 12, wherein the formulation comprises:
i. the weight ratio of the ginger composition to bisabolol and extracts containing bisabolol ranges from 1:10 to 1:100,000;
ii. the combined content of bisabolol and the ginger composition is at least 90 wt-% of the formulation;
iii. the ginger composition comprises 0.001-10 wt-% of the formulation;
iv. the bisabolol comprises 90-99.999 wt-% of the formulation; or
v. a combination of (i)-(iv).

22. The method of claim 12, further comprising administering a mist of the formulation to a person in need thereof, wherein at least 15 wt-% of the active mixture is delivered to the lower portions of the respiratory tract.
